SEQUENCE LISTING (16 sequences)

SEQ ID NO: 1 - protein, 227 aa, Homo sapiens
YPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 2 - protein, 76 aa, Homo sapiens
RHTRSAEDDSERDPLNVLKPRARMTPAPASCSQELPSAEDNSPMASDPLGVVRGGRVNTHAGGTGPEGCRPFAKFI

SEQ ID NO: 3 - protein, 72 aa, Homo sapiens
SAEDDSERDPLNVLKPRARMTPAPASCSQELPSAEDNSPMASDPLGVVRGGRVNTHAGGTGPEGCRPFAKFI

SEQ ID NO: 4 - protein, 68 aa, Homo sapiens
DSERDPLNVLKPRARMTPAPASCSQELPSAEDNSPMASDPLGVVRGGRVNTHAGGTGPEGCRPFAKFI

SEQ ID NO: 5 - protein, 46 aa, Homo sapiens
CSQELPSAEDNSPMASDPLGVVRGGRVNTHAGGTGPEGCRPFAKFI

SEQ ID NO: 6 - protein, 26 aa, Homo sapiens
SAEDDSERDPLNVLKPRARMTPAPAS

SEQ ID NO: 7 - protein, 33 aa, Artificial Sequence
  synthetically generated design sequence containing tag
GSSHHHHHHSSGLVPRGSHMASMTGGQQMGRGS

SEQ ID NO: 8 - protein, 30 aa, Homo sapiens
RHTRSAEDDSERDPLNVLKPRARMTPAPAS

SEQ ID NO: 9 - protein, 22 aa, Homo sapiens
DSERDPLNVLKPRARMTPAPAS

SEQ ID NO: 10 - protein, 46 aa, Homo sapiens; identical to SEQ ID NO: 5

SEQ ID NO: 11 - protein, 41 aa, Homo sapiens
PSAEDNSPMASDPLGVVRGGRVNTHAGGTGPEGCRPFAKFI

SEQ ID NO: 12 - protein, 35 aa, Homo sapiens
SPMASDPLGVVRGGRVNTHAGGTGPEGCRPFAKFI

SEQ ID NO: 13 - protein, 21 aa, Homo sapiens
RVNTHAGGTGPEGCRPFAKFI

SEQ ID NO: 14 - protein, 11 aa, Homo sapiens
PEGCRPFAKFI

SEQ ID NO: 15 - protein, 8 aa, Homo sapiens
CRPFAKFI

SEQ ID NO: 16 - DNA, 66 bp, Homo sapiens
gactcggagcgggaccccctgaacgtgctgaagccccgggcccggatgaccccggccccggcctcc

(19) United States
(12) Patent Application Publication
(10) Pub. No.: US 2010/0062984 A1
(43) Pub. Date: Mar. 11, 2010

(54) FGF-23 POLYPEPTIDES

(76) Inventors: Rajiv Kumar, Rochester, MN (US); Theresa J. Berndt, Rochester, MN (US); Theodore A. Craig, Rochester, MN (US)

Correspondence Address: FISH & RICHARDSON P.C., PO BOX 1022, MINNEAPOLIS, MN, 55440-1022, US

(21) Appl. No.: 12/523,026

(22) PCT Filed: Jan. 24, 2008
(86) PCT No.: PCT/US08/51935

§ 371 Date: Nov. 16, 2009

Related U.S. Application Data
(60) Provisional application No. 60/897,428, filed on Jan. 25, 2007.

(51) Int. Cl. A61K 38/16; C07K 14/00

(52) U.S. Cl. 514/13; 530/326

(57) ABSTRACT

This document relates to FGF-23 polypeptides, FGF-23 polypeptide fragments, and methods for making and using such polypeptides and polypeptide fragments. For example, FGF-23 polypeptide fragments that can be used to treat disorders associated with hyperphosphatemia such as chronic renal failure and hypoparathyroidism are provided.

### BACKGROUND

1. Technical Field

This document relates to FGF-23 polypeptides, polypeptide fragments of FGF-23 polypeptides, and methods for making and using such polypeptides and polypeptide fragments.

2. Background Information

Fibroblast growth factors (FGFs) are involved in many biological processes including embryonic development and cellular homeostasis in the adult (Cancilla et al., Kidney Int., 60:147-155, (2001) and Cancilla et al., Kidney Int., 56:2025-2039 (1999)). The FGFs are heparin-binding proteins, and interactions with cell-surface associated heparan sulfate proteoglycans have been reported to be important for FGF signal transduction. FGF molecules bind to a family of plasma membrane associated receptors, the fibroblast growth factor receptors (FGFR1, FGFR2, FGFR3, and FGFR4). Alternate mRNA splicing gives rise to distinct forms of FGFRs, which differ significantly in their ligand-binding profiles. The signaling complex at the cell surface is believed to be a ternary complex formed between two identical FGF ligands, two identical FGFR subunits, and either one or two heparan sulfate chains. In the case of FGF-23, a polypeptide called Klotho is believed to be involved in signal transduction. Twenty three fibroblast growth factors and four fibroblast growth factor receptors transduce a variety of biochemical changes in developing and adult tissues (Braun et al., Philos. Trans. R. Soc. Lond. B. Biol. Sci., 359:753-757 (2004); Steiling and Werner, Curr. Opin. Biotechnol., 14:533-537 (2003); and Eswarakumar et al., Cytokine Growth Factor Rev., 16:139-149 (2005)).

### SUMMARY

This document relates to FGF-23 polypeptides, polypeptide fragments of an FGF-23 polypeptide, and methods for making and using such polypeptides and polypeptide fragments. For example, this document provides polypeptide fragments of an FGF-23 polypeptide that can be used to treat disorders associated with hyperphosphatemia such as chronic renal failure and hypoparathyroidism.

In general, one aspect of this document features a substantially pure polypeptide comprising, or consisting essentially of, the sequence set forth in SEQ ID NO:2, SEQ ID NO:3, SEQ ID NO:4, SEQ ID NO:6, SEQ ID NO:8, or SEQ ID NO:9 and lacking the sequence set forth in SEQ ID NO:10, SEQ ID NO:11, SEQ ID NO:12, SEQ ID NO:13, or SEQ ID NO:14. The polypeptide can contain one or more modifications. The polypeptide can be covalently attached to one or more oligomers. The polypeptide can be pegylated.

In another aspect, this document features a composition comprising, or consisting essentially of, a polypeptide comprising, or consisting essentially of, the sequence set forth in SEQ ID NO:2, SEQ ID NO:3, SEQ ID NO:4, SEQ ID NO:6, SEQ ID NO:8, or SEQ ID NO:9 and lacking the sequence set forth in SEQ ID NO:10, SEQ ID NO:11, SEQ ID NO:12, SEQ ID NO:13, or SEQ ID NO:14. The composition can be formulated for oral or parenteral administration.

In another aspect, this document features a method for treating a disorder associated with hyperphosphatemia. The method comprises, or consists essentially of, administering a polypeptide to a mammal under conditions wherein the severity of a symptom of the disorder is reduced, wherein the polypeptide comprises, or consists essentially of, the sequence set forth in SEQ ID NO:2, SEQ ID NO:3, SEQ ID NO:4, SEQ ID NO:6, SEQ ID NO:8, or SEQ ID NO:9 and lacks the sequence set forth in SEQ ID NO:10, SEQ ID NO:11, SEQ ID NO:12, SEQ ID NO:13, or SEQ ID NO:14. The mammal can be a human. The disorder can be selected from the group consisting of chronic renal failure, hypoparathyroidism, metabolic acidosis, and respiratory acidosis. The polypeptide can be administered orally or parenterally.

In another aspect, this document features a method for reducing the serum phosphate level in a mammal. The method comprises, or consists essentially of, administering a polypeptide to a mammal under conditions wherein the serum phosphate level in the mammal is reduced, wherein the polypeptide comprises, or consists essentially of, the sequence set forth in SEQ ID NO:2, SEQ ID NO:3, SEQ ID NO:4, SEQ ID NO:6, SEQ ID NO:8, or SEQ ID NO:9 and lacks the sequence set forth in SEQ ID NO:10, SEQ ID NO:11, SEQ ID NO:12, SEQ ID NO:13, or SEQ ID NO:14.

In another aspect, this document features a method for increasing phosphate excretion in a mammal. The method comprises, or consists essentially of, administering a polypeptide to a mammal under conditions wherein phosphate excretion is increased in the mammal, wherein the polypeptide comprises, or consists essentially of, the sequence set forth in SEQ ID NO:2, SEQ ID NO:3, SEQ ID NO:4, SEQ ID NO:6, SEQ ID NO:8, or SEQ ID NO:9 and lacks the sequence set forth in SEQ ID NO:10, SEQ ID NO:11, SEQ ID NO:12, SEQ ID NO:13, or SEQ ID NO:14.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains graphs plotting excretion of sodium (FE Na) and phosphate (FE Pi) in rats infused with full length recombinant FGF-23 polypeptide, FGF-23₍₁₇₆₋₂₅₁₎ polypeptide, FGF-23₍₁₈₀₋₂₅₁₎ polypeptide, FGF-23₍₁₈₄₋₂₅₁₎ polypeptide, FGF-23₍₂₀₆₋₂₅₁₎ polypeptide, or FGF-23₍₁₈₀₋₂₀₅₎ polypeptide. The solute excretion in period C1 (control) was subtracted form the value in the experimental period (C2). An * indicates a significant difference, C1 compared to C2, paired T test, p<0.05. Data are expressed as mean±SEM.

FIG. 2 is a graph plotting serum phosphate levels of Fgf-23^{-/-} mice before and after i.p. injection of vehicle (saline), FGF-23 R176Q polypeptide, or FGF-23₍₁₈₀₋₂₀₅₎ polypeptide. NS=not significant; **=P<0.01.

FIG. 3 is a graph plotting percent phosphate uptake in opossum kidney (OK) cells that were mock-treated (control) or treated with the indicated amounts of FGF-23 polypeptide and FGF-23₍₁₈₀₋₂₅₁₎ polypeptide. An * indicates statistically significant differences, experimental vs. control, non-paired T test, p<0.05. Data are expressed as mean±SEM.

FIG. 4 contains fluorescence photomicrographs of OK cells expressing a V5-tagged Na⁺-Pi Ha transporter that were analyzed by immunohistochemistry for the presence of the transporter on the cell surface following treatment with FGF-23, FGF-23₍₁₈₀₋₂₅₁₎, parathyroid hormone₍₁₋₃₄₎ polypeptide (PTH₍₁₋₃₄₎ polypeptide), or PTH₍₁₃₋₃₄₎ polypeptide, or with a vehicle or antibody control.

### DETAILED DESCRIPTION

This document provides methods and materials related FGF-23 polypeptides, polypeptide fragments of an FGF-23 polypeptide, and methods for making and using such polypeptides and polypeptide fragments. An FGF-23 polypeptide can be from any species including, without limitation, dogs, cats, horses, bovine, sheep, monkeys, and humans. Amino acid sequences for FGF-23 polypeptides can be as set forth in GenBank gi accession numbers 10119774, 10119772, and 18543369 (see, also, accession numbers BAB13477, BAB13478, and NP_{—}570110). Nucleic acid sequences that encode an FGF-23 polypeptide can be as set forth in GenBank gi accession numbers 15055547, 110625790, and 18543368 (see, also, accession numbers NM_{—}020638, NM_{—}022657, and NM_{—}130754). A polypeptide fragment of an FGF-23 polypeptide can be any length, for example, between 22 amino acid residues and 76 amino acid residues (e.g., between 22 and 75 amino acid residues, between 22 and 70 amino acid residues, between 30 and 76 amino acid residues, or between 35 and 76 amino acid residues). Examples of such fragments include the polypeptides set forth in Table 1 and SEQ ID NOs:2-4, SEQ ID NO:6, and SEQ ID NOs:8-9.

TABLE 1
Fragments of FGF-23 polypeptides

| Name of | |
|---|---|
| Polypeptide | Amino Acid Sequence |
| FGF-23₍₁₇₆₋₂₅₁₎ | 176-RHTRSAEDDSERDPLNVLKPRARMTPAPAS |
| | CSQELPSAEDNSPMASDPLGVVRGGRVNTHAGGT |
| | GPEGCRPFAKFI-251 (SEQ ID NO: 2) |
| FGF-23₍₁₈₀₋₂₅₁₎ | 180-SAEDDSERDPLNVLKPRARMTPAPASCSQE |
| | LPSAEDNSPMASDPLGVVRGGRVNTHAGGTGPEG |
| | CRPFAKFI-251 (SEQ ID NO: 3) |
| FGF-23₍₁₈₄₋₂₅₁₎ | 184-DSERDPLNVLKPRARMTPAPASCSQELPSA |
| | EDNSPMASDPLGVVRGGRVNTHAGGTGPEGCRPF |
| | AKFI-251 (SEQ ID NO: 4) |
| FGF-23₍₁₈₀₋₂₀₅₎ | 180-SAEDDSERDPLNVLKPRARMTPAPAS-205 |
| | (SEQ ID NO: 6) |
| FGF-23₍₁₇₆₋₂₀₅₎ | 176-RHTRSAEDDSERDPLNVLKPRARMTPAP |
| | AS-205 (SEQ ID NO: 8) |
| FGF-23₍₁₈₄₋₂₀₅₎ | 184-DSERDPLNVLKPRARMTPAPAS-205 |
| | (SEQ ID NO: 9) |

A polypeptide fragment of an FGF-23 polypeptide can lack an amino acid sequence set forth in Table 2. For example, an FGF-23 polypeptide fragment can have the amino acid sequence set forth in SEQ ID NO:8 and lack the amino acid sequence set forth in any of SEQ ID NOs:10-15. In some cases, an FGF-23 polypeptide fragment can have the amino acid sequence set forth in SEQ ID NO:9 and lack the amino acid sequence set forth in any of SEQ ID NOs:10-15.

TABLE 2
Amino acid sequences

| SEQ ID | |
|---|---|
| NO: | Amino Acid Sequence |
| 10 | CSQELPSAEDNSPMASDPLGVVRGGRVNTHAGGTGPEGCRPFA |
| | KFI |
| 11 | PSAEDNSPMASDPLGVVRGGRVNTHAGGTGPEGCRPFAKFI |
| 12 | SPMASDPLGVVRGGRVNTHAGGTGPEGCRPFAKFI |
| 13 | RVNTHAGGTGPEGCRPFAKFI |
| 14 | PEGCRPFAKFI |
| 15 | CRPFAKFI |

The polypeptides and polypeptide fragments provided herein can be substantially pure. The term “substantially pure” as used herein with reference to a polypeptide means the polypeptide is substantially free of other polypeptides, lipids, carbohydrates, and nucleic acid with which it is naturally associated. A substantially pure polypeptide can be any polypeptide that is removed from its natural environment and is at least 60 percent pure. A substantially pure polypeptide can be at least about 65, 70, 75, 80, 85, 90, 95, or 99 percent pure. Typically, a substantially pure polypeptide will yield a single major band on a non-reducing polyacrylamide gel. A substantially pure polypeptide can be a chemically synthesized polypeptide.

Any method can be used to obtain a substantially pure polypeptide provided herein. For example, common polypeptide purification techniques such as affinity chromotography and HPLC as well as polypeptide synthesis techniques can be used to obtain an FGF-23 polypeptide or a fragment of an FGF-23 polypeptide (e.g., an FGF-23₍₁₇₆₋₂₀₅₎ polypeptide). In addition, any material can be used as a source to obtain a substantially pure polypeptide. For example, tissue from wild-type or transgenic animals can be used as a source material. In some cases, tissue culture cells engineered to over-express a particular polypeptide can be used to obtain substantially pure polypeptide. In some cases, a polypeptide can be engineered to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini, or in between. Other fusions that can be used include enzymes that aid in the detection of the polypeptide, such as alkaline phosphatase.

An FGF-23 polypeptide or polypeptide fragment provided herein can contain one or more modifications. For example, an FGF-23 polypeptide fragment can be modified to be pegylated or acylated, or to contain additional amino acid sequences such as an albumin sequence (e.g., a human albumin sequence). In some cases, an FGF-23 polypeptide fragment can be a fusion polypeptide that contains a fragment of an albumin sequence (e.g., a human albumin sequence). In some cases, an FGF-23 polypeptide fragment can be covalently attached to oligomers, such as short, amphiphilic oligomers that enable oral administration or improve the pharmacokinetic or pharmacodynamic profile of a conjugated FGF-23 polypeptide fragment. The oligomers can comprise water soluble PEG (polyethylene glycol) and lipid soluble alkyls (short chain fatty acid polymers). See, for example, International Patent Application Publication No. WO 2004/047871. In some cases, an FGF-23 polypeptide fragment can be fused to the Fc domain of an immunoglobulin molecule (e.g., an IgG1 molecule) such that active transport of the fusion polypeptide across epithelial cell barriers via the Fc receptor occurs. In some cases, a polypeptide provided herein can contain chemical structures such as 8-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides. In some cases, a polypeptide provided herein such as a polypeptide fragments provided herein (e.g., an FGF-23₍₁₇₆₋₂₀₅₎ polypeptide) can be a cyclic polypeptide.

A polypeptide provided herein can contain one or more amino acid additions, subtractions, or substitutions relative to another polypeptide (e.g., a wild-type FGF23 polypeptide). Such polypeptides can be prepared and modified as described herein. Amino acid substitutions can be conservative amino acid substitutions. Conservative amino acid substitutions are, for example, aspartic acid/glutamic acid as acidic amino acids; lysine/arginine/histidine as basic amino acids; leucine/isoleucine, methionine/valine, alanine/valine as hydrophobic amino acids; and serine/glycine/alanine/threonine as hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide.

In some cases, amino acid substitutions can be substitutions that do not differ significantly in their effect on maintaining (a) the structure of the polypeptide backbone in the area of the substitution, (b) the charge or hydrophobicity of the molecule at the target site, or (c) the bulk of the side chain. Naturally occurring residues are divided into groups based on common side-chain properties: (1) hydrophobic: norleucine, methionine, alanine, valine, leucine, isoleucine; (2) neutral hydrophilic: cysteine, serine, threonine; (3) acidic: aspartic acid, glutamic acid; (4) basic: asparagine, glutamine, histidine, lysine, arginine; (5) residues that influence chain orientation: glycine, proline; and (6) aromatic; tryptophan, tyrosine, phenylalanine. In some cases, non-conservative substitutions can be used. A non-conservative substitution can include exchanging a member of one of the classes described herein for another.

This document also provides nucleic acid molecules having a sequence that encodes any of the polypeptides provided herein. For example, this document provides nucleic acid molecules that encode an FGF-23 polypeptide or a polypeptide fragment of an FGF-23 polypeptide. In some cases, a nucleic acid molecule provided herein can encode an FGF-23₍₁₇₆₋₂₀₅₎ polypeptide.

The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

In some cases, an isolated nucleic acid molecules provided herein can be at least about 12 bases in length (e.g., at least about 13, 14, 15, 16, 17, 18, 19, 20, 25, 30, 40, 50, 60, 100, 250, 500, 750, 1000, 1500, 2000, 3000, 4000, or 5000 bases in length) and hybridize, under hybridization conditions, to the sense or antisense strand of a nucleic acid having a sequence that encodes the sequence set forth in SEQ ID NO:9. Such a nucleic acid can contain the following nucleic acid sequence: 5′-GACTCGGAGCGG-GACCCCCTGAACGTGCTGAAGCCCCGGGCCCGGATGACCCCGGCCCCGGCCT CC-3′ (SEQ ID NO:16). The hybridization conditions can be moderately or highly stringent hybridization conditions. In some cases, such nucleic acid molecules can be molecules that do not hybridize to the sense or antisense strand of a nucleic acid that consists only of the nucleic acid sequence that encodes the amino acid sequence set forth in SEQ ID NO:10, 11, 12, 13, 14, or 15. Examples of nucleic acid sequences that encode the amino acid sequence set forth in SEQ ID NO:10, 11, 12, 13, 14, or 15 are located at the appropriate portion of the 3′ end of the sequence set forth in GenBank® gi number 10119773 (see, also, GenBank® accession number AB037973).

For the purpose of this document, moderately stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/m), while the washes are performed at about 50° C. with a wash solution containing 2×SSC and 0.1% sodium dodecyl sulfate.

Highly stringent hybridization conditions mean the hybridization is performed at about 42° C. in a hybridization solution containing 25 mM KPO₄ (pH 7.4), 5×SSC, 5×Denhart's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

An isolated nucleic acid molecule provided herein can be obtained using any method including, without limitation, common molecular cloning and chemical nucleic acid synthesis techniques. For example, PCR can be used to obtain an isolated nucleic acid molecule containing a nucleic acid sequence sharing similarity to sequences that encode the amino acid sequence set forth in SEQ ID NO:9. PCR refers to a procedure or technique in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complementary DNA strands.

An isolated nucleic acid molecule provided herein can be obtained by mutagenesis. For example, an isolated nucleic acid containing a sequence that encodes the amino acid sequence set forth in SEQ ID NO:9 can be mutated using common molecular cloning techniques (e.g., site-directed mutagenesis). Possible mutations include, without limitation, additions, subtractions, and substitutions, as well as combinations of additions, subtractions, and substitutions.

This document also provides host cells containing at least one of the isolated nucleic acid molecules provided herein. Such cells can be prokaryotic and eukaryotic cells. It is noted that cells containing an isolated nucleic acid molecule provided herein are not required to express a polypeptide. In addition, the isolated nucleic acid molecule can be integrated into the genome of the cell or maintained in an episomal state. Thus, host cells can be stably or transiently transfected with a construct containing an isolated nucleic acid molecule provided herein.

Host cells can contain an exogenous nucleic acid molecule that encodes a polypeptide provided herein (e.g., a polypeptide containing an amino acid sequence as set forth in SEQ ID NO:9). Such host cells can express the encoded polypeptide.

Any methods can be used to introduce an isolated nucleic acid molecule into a cell in vivo or in vitro. For example, calcium phosphate precipitation, electroporation, heat shock, lipofection, microinjection, and viral-mediated nucleic acid transfer are common methods that can be used to introduce an isolated nucleic acid molecule into a cell. In addition, naked DNA can be delivered directly to cells in vivo as describe elsewhere (U.S. Pat. No. 5,580,859 and U.S. Pat. No. 5,589,466 including continuations thereof). Further, isolated nucleic acid molecules can be introduced into cells by generating transgenic animals.

Any methods can be used to identify cells containing an isolated nucleic acid molecule provided herein. Such methods include, without limitation, PCR and nucleic acid hybridization techniques such as Northern and Southern analysis. In some cases, immunohistochemistry and biochemical techniques can be used to determine if a cell contains a particular isolated nucleic acid molecule by detecting the expression of a polypeptide encoded by that particular nucleic acid molecule.

As described herein, the polypeptides provided herein as well as compositions containing one or more of such polypeptides can be used to treat a disorder associated with hyperphosphatemia. Any route of administration (e.g., oral or parenteral administration) can be used to administer a polypeptide or composition provided herein (e.g., a composition containing one or more of the polypeptides provided herein) to a mammal. For example, a composition can be administered orally or parenterally (e.g., a subcutaneous, intramuscular, intraorbital, intracapsular, intraspinal, intrasternal, or intravenous injection). Compositions containing a polypeptide provided herein can contain additional ingredients such as those described in U.S. Pat. No. 6,818,619. Such additional ingredients can be polypeptides or non-polypeptides (e.g., buffers). In addition, the polypeptides within a composition provided herein can be in any form such as those described in U.S. Pat. No. 6,818,619.

Before administering a composition provided herein to a mammal, the mammal can be assessed to determine whether or not the mammal has a need for a treatment of a disorder associated with hyperphosphatemia. After identifying a mammal as having a need for a treatment of a disorder associated with hyperphosphatemia, the mammal can be treated with a composition provided herein. For example, a composition containing one or more polypeptides provided herein can be administered to a mammal in any amount, at any frequency, and for any duration effective to achieve a desired outcome (e.g., to reduce serum phosphate levels in a mammal, to increase phosphate excretion in a mammal, or to treat a disorder associated with hyperphosphatemia).

An effective amount of a composition can be any amount that reduces serum phosphate levels in a mammal, increases phosphate excretion in a mammal, or treats a disorder associated with hyperphosphatemia in a mammal without producing significant toxicity to the mammal. If a particular mammal fails to respond to a particular amount, then the amount can be increased by, for example, ten fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment.

The frequency of administration can be any frequency that that reduces serum phosphate levels in a mammal, increases phosphate excretion in a mammal, or treats a disorder associated with hyperphosphatemia in a mammal without producing significant toxicity to the mammal. For example, the frequency of administration can be from about four times a day to about once every other month, or from about once a day to about once a month, or from about once every other day to about once a week. In addition, the frequency of administration can remain constant or can be variable during the duration of treatment. As with the effective amount, various factors can influence the actual frequency of administration used for a particular application. For example, the effective amount, duration of treatment, route of administration, and severity of the disorder associated with hyperphosphatemia may require an increase or decrease in administration frequency.

An effective duration of administration can be any duration that reduces serum phosphate levels in a mammal, increases phosphate excretion in a mammal, or treats a disorder associated with hyperphosphatemia in a mammal without producing significant toxicity to the mammal. The effective duration can vary from several days to several weeks, months, or years. In general, the effective duration can range in duration from several days to several months. Typically, an effective duration can range from about one to two weeks to about 36 months. Prophylactic treatments can be typically longer in duration and can last throughout an individual mammal's lifetime.

Multiple factors can influence the actual effective duration used for a particular treatment or prevention regimen. For example, an effective duration can vary with the frequency of administration, amount administered, route of administration, and severity of the disorder associated with hyperphosphatemia.

After administering a composition provided herein to a mammal, the mammal can be monitored to determine whether or not serum phosphate concentrations have decreased.

In some cases, after administering a composition provided herein to a mammal, the mammal can be monitored to determine the serum level of a polypeptide provided herein. Any appropriate method can be used to measure serum levels of a polypeptide provided herein including, without limitation, mass spectrometry and immunological methods such as ELISA. An antibody used in an immunological assay can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type, (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. Such an antibody can be capable of binding specifically to a polypeptide provided herein.

Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. In some cases, an antibody fragment can be enzymatically or chemically produced by fragmentation of an intact antibody. An antibody directed against a polypeptide provided herein can bind the polypeptide at an affinity of at least 10⁴ mol⁻¹ (e.g., at least 10⁵, 10⁶, 10⁷, 10⁸, 10⁹, 10¹⁰, 10¹¹, or 10¹² mol⁻¹).

The invention will be further described in the following examples, which do not limit the scope of the invention described in the claims.

### EXAMPLES

### Example 1

### Protein and Peptide Synthesis

Synthesis of full-length recombinant human FGF-23₍₂₅₋₂₅₁₎ polypeptide was carried out using bacterial protein expression methods. Human FGF-23₍₂₅₋₂₅₁₎ polypeptide was expressed in pET28a(+), E. coli Rosetta 2(DE3) cells (Novagen/EMD, San Diego, Calif.), at 20° C. in the presence of the inducer, isopropyl β-D-thiogalactoside (IPTG; 0.1 mM). Cells were lysed at 4° C. in lysis buffer (20 mM Na₂HPO₄, 1.0 M NaCl, 10 mM beta-mercaptoethanol, pH 7.0) with 4 mM phenylmethanesulfonyl fluoride (PMSF) using an ice jacketed Bead Beater (Biospec Industries, Inc., Bartlesville, Okla.; eight cycles of 20 seconds on/ two minutes off). The cell lysate was centrifuged at 20,000×g for 30 minutes at 4° C. The supernatant was stirred at 4° C. with 15 mL nickel chelating sepharose (Amersham/GE Healthcare Bio-Sciences Corp., Piscataway, N.J.) for two hours. The resin was pelleted by centrifugation, extensively washed, and then finally washed on a column with one liter lysis buffer at 4° C. over 12 hours. Protein was eluted with lysis buffer containing 1 M imidazole (80 mL) and dialyzed against lysis buffer before being loaded onto a five mL HisTrap column (Amersham/GE Healthcare). Protein was eluted with an imidazole gradient (0-1 M) in lysis buffer. Fractions were analyzed by SDS-PAGE and Coomassie blue/silver stain and by immunoblots (PhastTransfer; Amersham/GE Healthcare) using an affinity purified polyclonal antibody against human FGF-23 polypeptide, and a goat anti-rabbit HRP secondary antibody (Dako, Carpinteria, Calif.), visualized by chemiluminescence (Roche, Indianapolis, Ind.). Fractions containing N-terminal 6×His human FGF-23₍₂₅₋₂₅₁₎ polypeptide were combined and dialyzed against 20 mM Na₂HPO₄, 5 mM beta-mercaptoethanol, pH 7.0. The dialyzed fractions were loaded onto an HR5/5 Mono S column (Amersham/GE Healthcare) and eluted with a NaCl gradient (0-1 M). Mono S fractions were analyzed by SDS-PAGE/immunobloting. Fractions containing highly purified human FGF-23₍₂₅₋₂₅₁₎ polypeptide (about 0.25 M NaCl) were pooled and further analyzed by N-terminal protein sequencing. FGF-23₍₂₅₋₂₅₁₎ polypeptide was dialyzed against N₂-purged 0.9% NaCl at 4° C. before animal infusions.

The synthesis of five FGF-23 polypeptides (Table 3) was performed by the Mayo Peptide Synthesis Facility (Mayo Clinic, Rochester, Minn.) by solid phase methods on an ABI 433A peptide synthesizer (Applied Biosystems Inc., Foster City, Calif.) using protocols described elsewhere for the synthesis of human amyloid-β peptides (Poduslo et al., Biochemistry, 43:6064-6075 (2004)). Briefly, FGF-23 polypeptides (0.1 mmol scale) were synthesized on NovaSyn TGA resin (Calbiochem-Novabiochem, San Diego, Calif.) using HBTU activation of N^{α}-9-fluoroenylmethoxy-carbonyl (Fmoc) amino acid derivatives and synthesis protocols provided by the instrument's manufacturer. After completion of synthesis, all polypeptides were cleaved from the TGA resin support using 10 mL of a solution of 87.5% trifluoroacetic acid (TFA), 5% water, 5% phenol, and 2.5% triisopropylsilane (v/v/wt/v) for two hours at 22° C. The cleaved peptides were then precipitated in 40 mL cold tert-butylmethyl ether and purified by reverse phase HPLC on a C₁₈ Jupiter column (250 mm×21.2 mm, Phenomenex Corp.) using a binary gradient of 0.1% aqueous TFA containing 5% acetonitrile (buffer A) and 0.1% aqueous TFA containing 80% acetonitrile (buffer B). The calculated mass for each FGF-23 synthetic polypeptide was verified by electrospray ionization mass spectrometry using a MSQ single quadrupole mass analyzer (Thermo Electron Corp., San Jose, Calif.) using the following parameters: start m/z of 500, stop m/z of 2000, dwell time of 0.1 msec at 40 scans/minute. Each spectrum was deconvoluted into a single mass using Bioworks Browser provided by Thermo Electron. All polypeptides were of the appropriate molecular mass (Table 3).

TABLE 3
Sequences and molecular weights of FGF-23 polypeptides

| | | |
|---|---|---|
| Name of | | |
| Polypeptide | Amino Acid Sequence | Molecular Weight |

| | | |
|---|---|---|
| Full-length | 25-YPNASPLLGSSWGGLIHLYTA | 25331 |
| FGF-23₍₂₅₋₂₅₁₎ | TARNSYHLQIHKNGHVDGAPHQ | 28744 with tag from |
| | TIYSALMIRSEDAGFVVITGVMSR | construct: GSSHHHH |
| | RYLCMDFRGNIFGSHYFDPENCR | HHSSGLVPRGSHMA |
| | FQHQTLENGYDVYHSPQYHFLVS | SMTGGQQMGRGS |
| | LGRAKRAFLPGMNPPPYSQFLSR | (SEQ ID NO: 7) |
| | RNEIPLIHFNTPIPRRHTRSAEDDS | |
| | ERDPLNVLKPRARMTPAPASCSQ | |
| | ELPSAEDNSPMASDPLGVVRGGR | |
| | VNTHAGGTGPEGCRPFAKFI-251 | |
| | (SEQ ID NO: 1) | |
| FGF-23₍₁₇₆₋₂₅₁₎ | 176-RHTRSAEDDSERDPLNVLKPR | 8070 |
| | ARMTPAPASCSQELPSAEDNSPMA | |
| | SDPLGVVRGGRVNTHAGGTGPEG | |
| | CRPFAKFI-251 | |
| | (SEQ ID NO: 2) | |
| FGF-23₍₁₈₀₋₂₅₁₎ | 180-SAEDDSERDPLNVLKPRARMT | 7519 |
| | PAPASCSQELPSAEDNSPMASDPL | |
| | GVVRGGRVNTHAGGTGPEGCRPF | |
| | AKFI-251 | |
| | (SEQ ID NO: 3) | |
| FGF-23₍₁₈₄₋₂₅₁₎ | 184-DSERDPLNVLKPRARMTPAPA | 7117 |
| | SCSQELPSAEDNSPMASDPLGVVR | |
| | GGRVNTHAGGTGPEGCRPFAKFI-251 | |
| | (SEQ ID NO: 4) | |
| FGF-23₍₂₀₆₋₂₅₁₎ | 206-CSQELPSAEDNSPMASDPLGV | 4714 |
| | VRGGRVNTHAGGTGPEGCRPFAK | |
| | FI-251 | |
| | (SEQ ID NO: 5) | |
| FGF-23₍₁₈₀₋₂₀₅₎ | 180-SAEDDSERDPLNVLKPRARMT | 2824 |
| | PAPAS-205 | |
| | (SEQ ID NO: 6) | |

### Example 2

### Animal Studies

Male Sprague Dawley rats weighing 300 g were purchased from Harlan Sprague Dawley Inc. (Madison Wis.). They were fed a standard rodent diet containing 0.7% phosphate and 0.5% calcium. All animals were fasted overnight prior to the experiment. On the day of the experiment, rats were anesthetized with an intraperitoneal injection of 100-150 mg/kg body weight of 5-sec-butyl-ethyl-2-thiobarbituricacid (Inactin, Byk Gulden Konstanz, Hamburg, Germany). The rats were placed on a heated table to maintain body temperature between 36-38° C. After a tracheostomy, a PE 50 catheter was placed in the left carotid artery to monitor mean arterial blood pressure (MAP) and to collect blood samples. Catheters were also placed in the left and right jugular veins for infusions. A solution containing 2% inulin in 0.9% NaCl plus 4.5% BSA was infused at a rate of 1.2 mL/hour in one catheter, and 0.9% NaCl was infused in the other catheter at a rate of 1.2 mL/hour. A catheter (PE 90) was placed in the bladder for urine collection. Acute infusion studies were performed using the following seven different groups of animals.

Group 1: Vehicle Time control (n=8). After a ninety-minute recovery period, one thirty-minute urine sample was taken (C1) and a blood sample was taken. One hour later a second 60-minute clearance was taken (C2).

Group 2: Effect of acute infusion of full length FGF-23 polypeptide (1.24 nmol/kg/hour, n=8). This protocol is identical to group 1 except that after the control collections, full-length recombinant FGF-23 polypeptide was added to the 0.9% saline infusion to deliver 1.24 nmol/kg/hour of full length FGF-23 polypeptide. After one hour of infusion, a sixty-minute clearance was taken.

Group 3: Effect of acute infusion of FGF-23₍₁₇₆₋₂₅₁₎ polypeptide (1.24 nmol/kg/hour, n=8). This protocol is identical to group 2 except for the sequence of the infused polypeptide.

Group 4: Effect of acute infusion of FGF-23₍₁₈₀₋₂₅₁₎ polypeptide (1.24 nmol/kg/hour, n=8). This protocol is identical to group 2 except for the sequence of the infused polypeptide.

Group 5: Effect of infusion of FGF-23₍₁₈₄₋₂₅₁₎ polypeptide (1.24 nmol/kg/hour, n=8). This protocol is identical to group 2 except for the sequence of the infused polypeptide.

Group 6: Effect of infusion of FGF-23₍₂₀₆₋₂₅₁₎ polypeptide (1.24 nmol/kg/hour, n=6). This protocol is identical to group 2 except for the sequence of the infused polypeptide.

Group 7: Effect of infusion of FGF-23₍₁₈₀₋₂₀₅₎ polypeptide (1.24 nmol/kg/hour, n=6). This protocol is identical to group 2 except for the sequence of the infused polypeptide.

Plasma and urine phosphate concentrations were determined using the method of Chen (Chen et al., Anal Chem., 28:1756-1758 (1956)). Inulin concentrations in plasma and urine were determined using the anthrone method (Führ et al., Klin. Wochenschr., 33.:729-730 (1955)). Sodium concentrations in urine were determined by ion selective electrode (EasyLyte Plus Analyzer, Medica Corp., Bedford, Mass.). Calcium concentrations were measured using atomic absorption spectrometry (Kumar et al., Biochemistry, 15:2420-2423 (1976)) with a Perkin Elmer Model 3300 Atomic Absorption Spectrometer (Perkin Elmer, Boston, Mass.). Serum 1α,25(OH)₂D concentrations were measured using an RIA kit (Diasorin, Stillwater, Minn.).

Statistically significant differences between groups were evaluated by Student's t-test for comparison between two groups or by one-way analysis of variance (ANOVA) followed by Tukey's test for multiple comparisons. All values were expressed as mean±SE. A p value less than 0.05 was considered to be statistically significant.

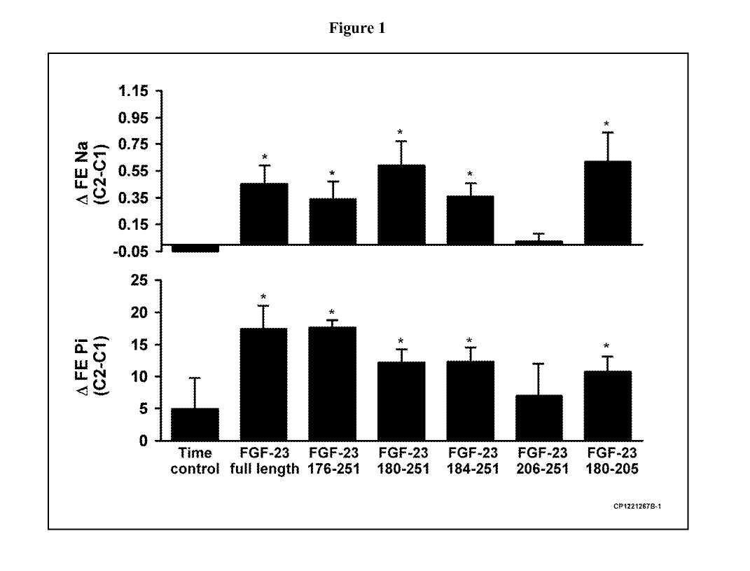

Data for groups 1-7, performed to determine the effect of the acute intravenous infusion of equimolar amounts of FGF-23 fragments in normal rats are summarized in Table 4. The glomerular filtration rate (GFR) was stable throughout the experiment in all groups studied. In the vehicle-infused group, the fractional excretion of sodium (FE Na) and fractional excretion of phosphate (FE Pi) were stable throughout the experiment (FIG. 1). The acute intravenous infusion of full length recombinant FGF-23 polypeptide significantly increased the FE Pi from 14±3 to 32±5% (p<0.001) and FE Na from 0.3±0.1 to 0.7±0.2% (p=0.01). FE Ca increased from 4.1±1.2% to 10.0±2.3% (p=0.039). Infusion of FGF-23₍₁₇₆₋₂₅₁₎ polypeptide significantly increased the FE Pi from 15±2 to 33±2% (p<0.0001) and the FE Na from 0.90±0.15 to 1.21±0.17 (p=0.020). Infusion of FGF-23₍₁₈₀₋₂₅₁₎ polypeptide significantly increased the FE Pi from 14±3 to 26±3% (p=0.0004) and FE Na from 0.20±0.07 to 0.80±0.17% (p=0.014). Infusion of FGF-23₍₁₈₄₋₂₅₁₎ polypeptide significantly increased FE Pi from 16±3 to 28±3% (p=0.0006) and FE Na from 0.3±0.1 to 0.6±0.16%, p<0.05 (p=0.006). Likewise, infusion of FGF-23₍₁₈₀₋₂₀₅₎ polypeptide significantly increased FE Pi from 10±2 to 20±2% (p=0.004) and FE Na from 0.4±0.1 to 1.0±0.2%, (p=0.03). In contrast, infusion of FGF-23₍₂₀₆₋₂₅₁₎ polypeptide did not increase either phosphate or sodium excretion.

TABLE 4
Effect on renal function of infusing equimolar amounts of FGF-23 polypeptides

| | GFR (mL/min) | | FE_{Pi} (%) | | FE_{Na} (%) | | P_{Pi} (mM) | | MAP (mmHg) | |
|---|---|---|---|---|---|---|---|---|---|---|
| | C | E | C | E | C | E | C | E | C | E |
| Time Control | 3.1 ± 0.6 | 3.6 ± 0.5 | 21 ± 4 | 26 ± 4 | 0.15 ± 0.05 | 0.13 ± 0.04 | 1.32 ± 0.05 | 1.49 ± 0.07 | 141 ± 5 | 131 ± 5 * |
| (n = 8) | | | | | | | | | | |
| FGF-23 | 3.5 ± 0.3 | 3.3 ± 0.5 | 14 ± 3 | 32 ± 5 * | 0.26 ± 0.14 | 0.76 ± 0.15 * | 1.53 ± 0.08 | 1.49 ± 0.10 | 146 ± 4 | 143 ± 5 |
| Full Length | | | | | | | | | | |
| (n = 8) | | | | | | | | | | |
| FGF-23₍₁₇₆₋₂₅₁₎ | 3.5 ± 0.5 | 3.1 ± 0.4 | 15 ± 2 | 33 ± 2 * | 0.87 ± 0.15 | 1.21 ± 0.17 * | 1.91 ± 0.13 | 1.91 ± 0.14 | 117 ± 8 | 122 ± 8 |
| (n = 8) | | | | | | | | | | |
| FGF-23₍₁₈₀₋₂₅₁₎ | 4.5 ± 0.4 | 4.2 ± 0.4 | 14 ± 3 | 26 ± 3 * | 0.17 ± 0.07 | 0.76 ± 0.17 * | 1.51 ± 0.10 | 1.42 ± 0.09 | 150 ± 4 | 150 ± 5 |
| (n = 8) | | | | | | | | | | |
| FGF-23₍₁₈₄₋₂₅₁₎ | 4.1 ± 0.3 | 4.3 ± 0.3 | 16 ± 3 | 28 ± 3 * | 0.27 ± 0.09 | 0.63 ± 0.16 * | 1.58 ± 0.07 | 1.46 ± 0.06 * | 133 ± 6 | 128 ± 5 |
| (n = 8) | | | | | | | | | | |
| FGF-23₍₂₀₆₋₂₅₁₎ | 4.0 ± 0.2 | 3.1 ± 0.2 | 22 ± 5 | 29 ± 2 | 0.19 ± 0.04 | 0.22 ± 0.05 | 1.34 ± 0.06 | 1.46 ± 0.06 | 144 ± 4 | 126 ± 4 * |
| (n = 6) | | | | | | | | | | |
| FGF-23₍₁₈₀₋₂₀₅₎ | 4.0 ± 0.5 | 4.5 ± 0.3 | 10 ± 2 | 20 ± 2 * | 0.39 ± 0.11 | 1.01 ± 0.21 * | 1.52 ± 0.09 | 1.54 ± 0.07 | 151 ± 0 | 145 ± 7 |
| (n = 6) | | | | | | | | | | |

C = Control Clearance
E = Experimental Clearance
GFR = Glomerular Filtration Rate
FEPi = Fractional Excretion of Phosphate
FENa = Fractional Excretion of Sodium
PPi = Plasma Phosphate Concentration
MAP = Mean Arterial Pressure.
* Indicates a significant difference (p < 0.05 paired T test); data are expressed as mean ± SE

The results of these studies demonstrate that acute intravenous infusions of equimolar doses of full length FGF-23 polypeptide and FGF-23₍₁₇₆₋₂₅₁₎ polypeptide result in similar increases in phosphate excretion. Infusion of FGF-23₍₁₈₀₋₂₅₁₎, FGF-23₍₁₈₀₋₂₀₅₎, and FGF-23₍₁₈₄₋₂₅₁₎ polypeptides also increases phosphate excretion significantly. Of interest, these bioactive polypeptides also increase sodium excretion when administered over the short-term. With long term-administration, the sodium wasting is no longer seen.

Chronic animal studies were performed to determine the effect of chronic (72 hour) administration of FGF-23₍₁₇₆₋₂₅₁₎ polypeptide (1.24 nmol/kg/hour, n=5) or vehicle (PBS+0.1% BSA, n=6) on serum phosphate and 1α, 25 (OH)2 D concentrations. Rats received an intraperitoneal injection of FGF-23₍₁₇₆₋₂₅₁₎ polypeptide in 0.5 mL of normal saline every 12 hours for three days. At the end of three days, the animals were anesthetized and a terminal blood sample was taken.

Chronic (72 hour) administration of FGF-23₍₁₇₆₋₂₅₁₎ polypeptide or vehicle resulted in serum phosphate concentrations of 1.85±0.12 mM and 2.19±0.11 mM (p=0.062). FGF-23₍₁₇₆₋₂₅₁₎ treatment significantly decreased serum 1α,25(OH)2D concentrations (76±11 pg/mL) compared to the vehicle treated rats (114±7 pg/mL, p<0.05). FGF-23₍₁₇₆₋₂₅₁₎ polypeptide given intraperitoneally also reduced serum Pi concentrations in Fgf-23^{-/-} mice.

To further examine the in vivo phosphaturic activity of the smallest FGF-23 fragment, 0.2 nmoles of FGF-23₍₁₈₀₋₂₀₅₎ polypeptide were injected twice intraperitoneally into Fgf-23^{-/-} mice. FGF-23 R176Q polypeptide was similarly injected into the mutant mice as a positive control, and saline was injected as a vehicle control. Fgf-23^{-/-} animals were generated as reported elsewhere (Sitara et al., Matrix Biol., 23:421-432 (2004)). To determine the in vivo effects of the C-terminal fragment FGF23₍₁₈₀₋₂₀₅₎ in the absence of endogenous FGF-23 activity, equimolar amounts (0.2 nmoles) of FGF-23 R176Q polypeptide, FGF-23₍₁₈₀₋₂₀₅₎ polypeptide, and vehicle (saline) were injected twice intraperitoneally into Fgf-23^{-/-} mice at 0 and 8 hours. Blood was obtained by tail bleeding or cheek pouch puncture of 3- to 6-week-old Fgf-23^{-/-} animals, 8 hours after the second injection (at 16 hours). Serum levels of phosphate were determined in pre- and post-injection mice using the Stanbio LiquiUV kit (Stanbio Laboratory, Boerne, Tex.).

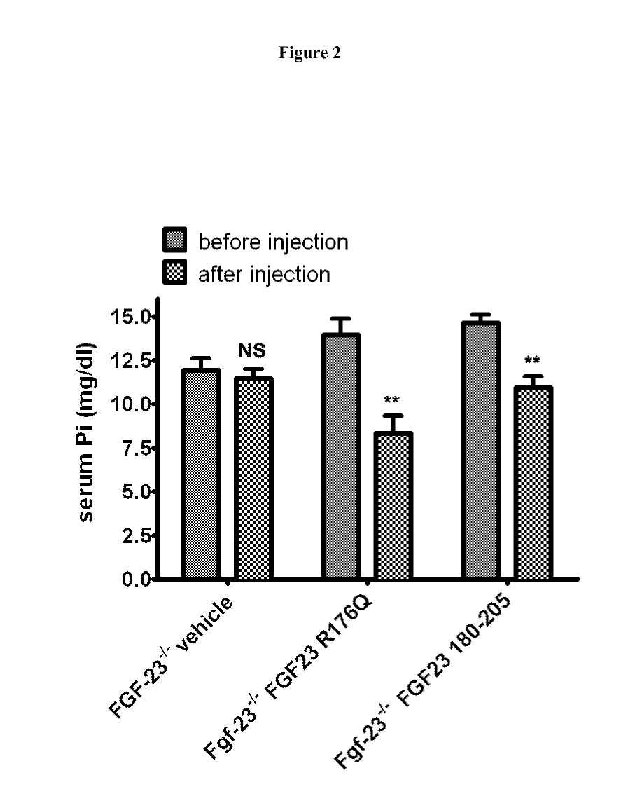

The results of these experiments indicate that the C-terminal FGF-23₍₁₈₀₋₂₀₅₎ polypeptide fragment was bioactive, as reflected by a significant decrease in serum phosphate levels in Fgf-23^{-/-} mice (14.64±0.51 pre-injection vs. 10.95±0.62 post-injection; p<0.01; FIG. 2). These data suggest that the C-terminal fragment of the FGF-23 polypeptide, containing only amino acid residues 180-205, could regulate phosphate homeostasis.

### Example 3

### In Vitro Studies

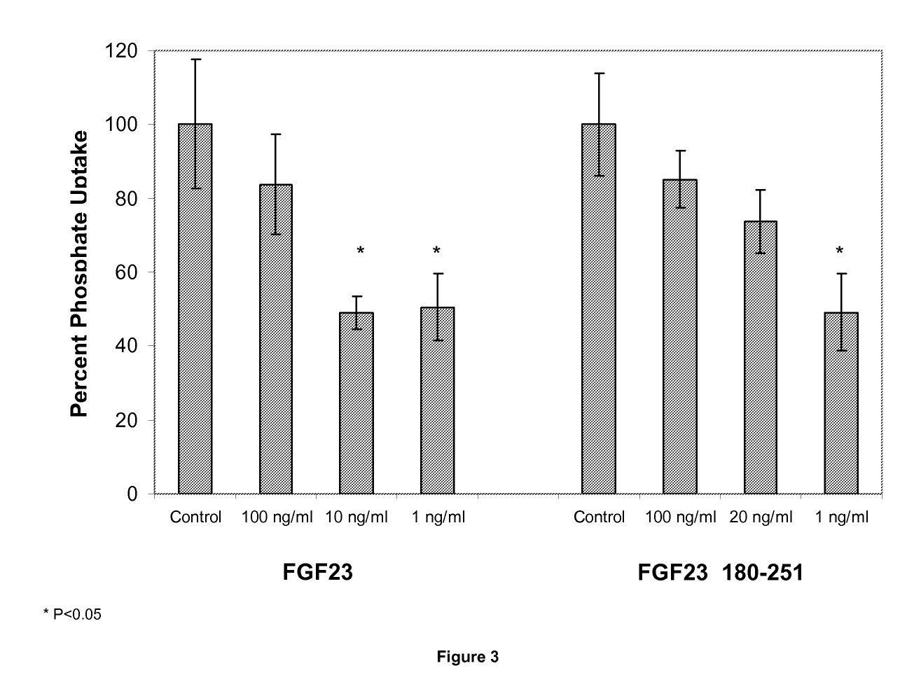

FGF-23 and FGF-23₍₁₈₀₋₂₅₁₎ polypeptide directly inhibited radiolabeled phosphorus uptake in the standard in vitro assay using opossum proximal tubular epithelial cells (OK; FIG. 3). The OK cell Pi uptake assay was performed as described by Bowe et al. (Biochem Biophys Res Commun., 284:977-981 (2001)).

To assess the mechanism by which these agents altered phosphate transport, the surface expression of an epitope tagged sodium phosphate transporter, NaPi IIa-V5, was examined. An OK cell line stably expressing a VS-tagged Na+-Pi IIa transporter was generated as described elsewhere (Berndt et al., Pflugers Arch., 451:579-587 (2006)). The presence of the transporter on the cell surface of OK-N+-Pi-IIa-V5 cells was carried out using immunohistochemical methods and reagents described elsewhere (Berndt et al., Pflugers Arch., 451:579-587 (2006)).

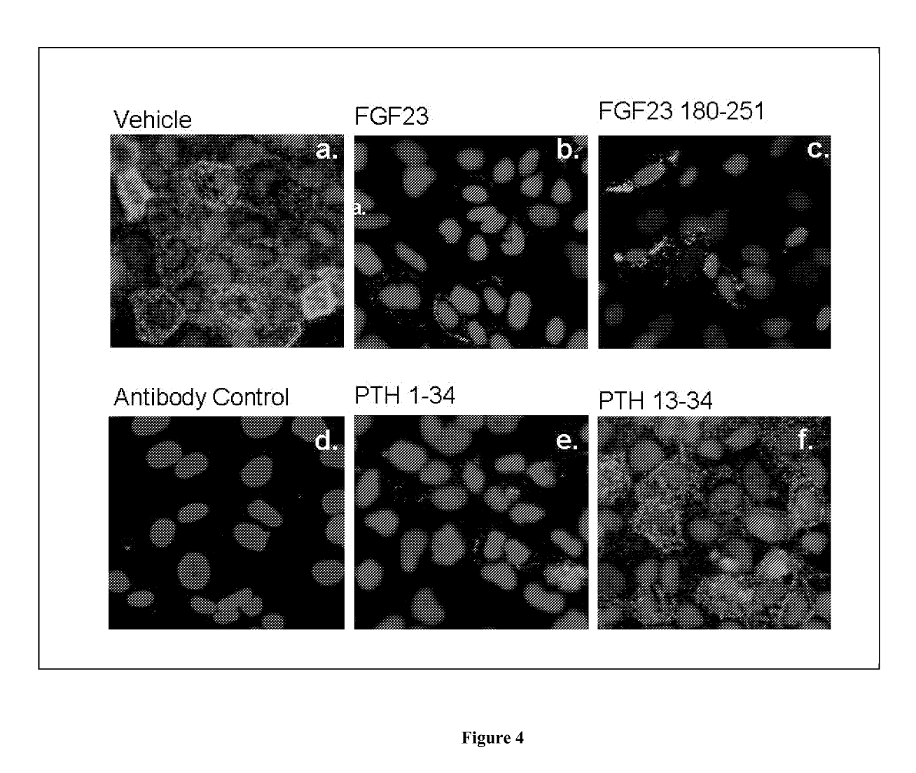

Immunofluorescent data confirmed that the anti-V5 antibody readily detected plasma membrane localized NaPi IIaV5 whereas there was no significant staining in the absence of the primary antibody (FIGS. 4a and 4d). Three hour treatment with FGF-23 or FGF-23₍₁₈₀₋₂₅₁₎ polypeptide resulted in significant decreases in the amount of NaPiIIa-V5 detected (FIGS. 4b and 4c). Similar decreases in immunodetection of NaPiIIa-V5 occurred after incubation with PTH 1-34 (FIG. 4e) but not the inactive PTH 13-34 (FIG. 40.

The in vitro studies demonstrate that similar to PTH, FGF-23 and FGF-23₍₁₈₀₋₂₅₁₎ polypeptide inhibit sodium-phosphate transport by reducing the amount of plasma membrane localized NaPi IIa.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A substantially pure polypeptide comprising the sequence set forth in SEQ ID NO:9 and lacking the sequence set forth in SEQ ID NO:10, SEQ ID NO:11, SEQ ID NO:12, SEQ ID NO:13, or SEQ ID NO:14.

2. The polypeptide of claim 1, wherein said polypeptide contains one or more modifications.

3. The polypeptide of claim 1, wherein said polypeptide is covalently attached to one or more oligomers.

4. The polypeptide of claim 1, wherein said polypeptide is pegylated.

5. A composition comprising a polypeptide comprising the sequence set forth in SEQ ID NO:9 and lacking the sequence set forth in SEQ ID NO:10, SEQ ID NO:11, SEQ ID NO:12, SEQ ID NO:13, or SEQ ID NO:14.

6. The composition of claim 5, wherein said composition is formulated for oral or parenteral administration.

7. A method for treating a disorder associated with hyperphosphatemia, said method comprising administering a polypeptide to a mammal under conditions wherein the severity of a symptom of said disorder is reduced, wherein said polypeptide comprises the sequence set forth in SEQ ID NO:9 and lacks the sequence set forth in SEQ ID NO:10, SEQ ID NO:11, SEQ ID NO:12, SEQ ID NO:13, or SEQ ID NO:14.

8. The method of claim 7, wherein said mammal is a human.

9. The method of claim 7, wherein said disorder is selected from the group consisting of chronic renal failure and hypoparathyroidism.

10. The method of claim 7, wherein said polypeptide is administered orally or parenterally.

11. A method for reducing the serum phosphate level in a mammal, said method comprising administering a polypeptide to a mammal under conditions wherein said serum phosphate level in said mammal is reduced, wherein said polypeptide comprises the sequence set forth in SEQ ID NO:9 and lacks the sequence set forth in SEQ ID NO:10, SEQ ID NO:11, SEQ ID NO:12, SEQ ID NO:13, or SEQ ID NO:14.

12. A method for increasing phosphate excretion in a mammal, said method comprising administering a polypeptide to a mammal under conditions wherein phosphate excretion is increased in said mammal, wherein said polypeptide comprises the sequence set forth in SEQ ID NO:9 and lacks the sequence set forth in SEQ ID NO:10, SEQ ID NO:11, SEQ ID NO:12, SEQ ID NO:13, or SEQ ID NO:14.
